(19) United States
(12) Patent Application Publication
(10) Pub. No.: US 2016/0038551 A1
(43) Pub. Date: Feb. 11, 2016

(54) STABILIZED MESEMBRINE COMPOSITIONS

(71) Applicant: BOTANICAL RESOURCE HOLDINGS PROPRIETARY LIMITED, Cape Town (ZA)

(72) Inventor: Richard Paul Davies, Somerset West (ZA)

(21) Appl. No.: 14/780,157

(22) PCT Filed: Mar. 28, 2014
(86) PCT No.: PCT/IB2014/060260

§ 371 Date: Sep. 25, 2015

(30) Foreign Application Priority Data
Mar. 28, 2013 (NA) 2013/0007

(51) Int. Cl. A61K 36/185; A61K 31/403

(52) U.S. Cl. CPC A61K 36/185; A61K 31/403

(57) ABSTRACT

A composition comprises as active ingredient an extract of a plant or plants from the Mesembryanthemaceae family, such as Mesembryanthemum tortuosum, having an alkaloid profile comprising at least 70% (w/w) stabilized mesembrine. The extracts from Mesembryanthemum tortuosum having an alkaloid profile comprising at least about 70% (w/w) stabilized mesembrine, about 20% (w/w) mesembrenone and about 10% (w/w) Δ7 mesembrenone act as bioamine releasing agents, in particular as monoamine releasing agents, useful in the treatment of diseases and conditions responsive to treatment with a monoamine releasing agent, for instance diseases and conditions responsive to treatment with a serotonin-releasing agent.

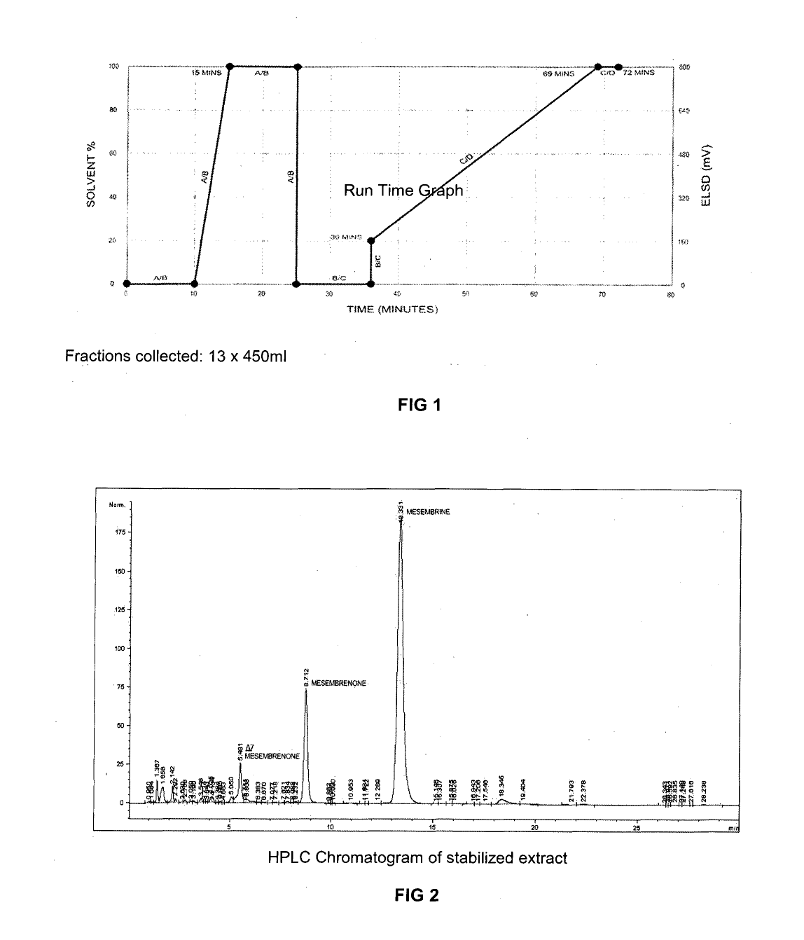

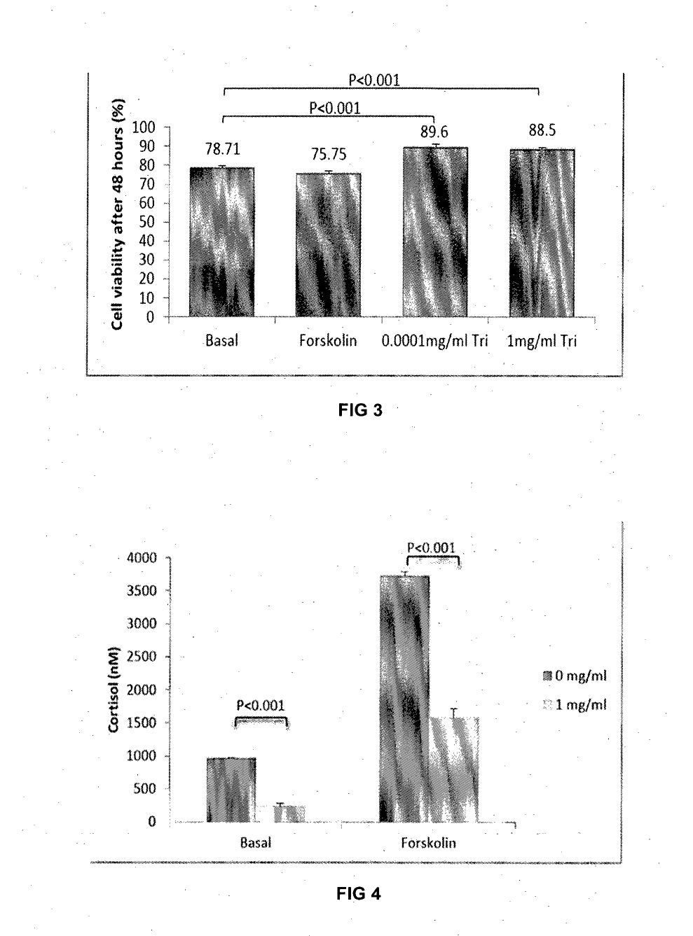

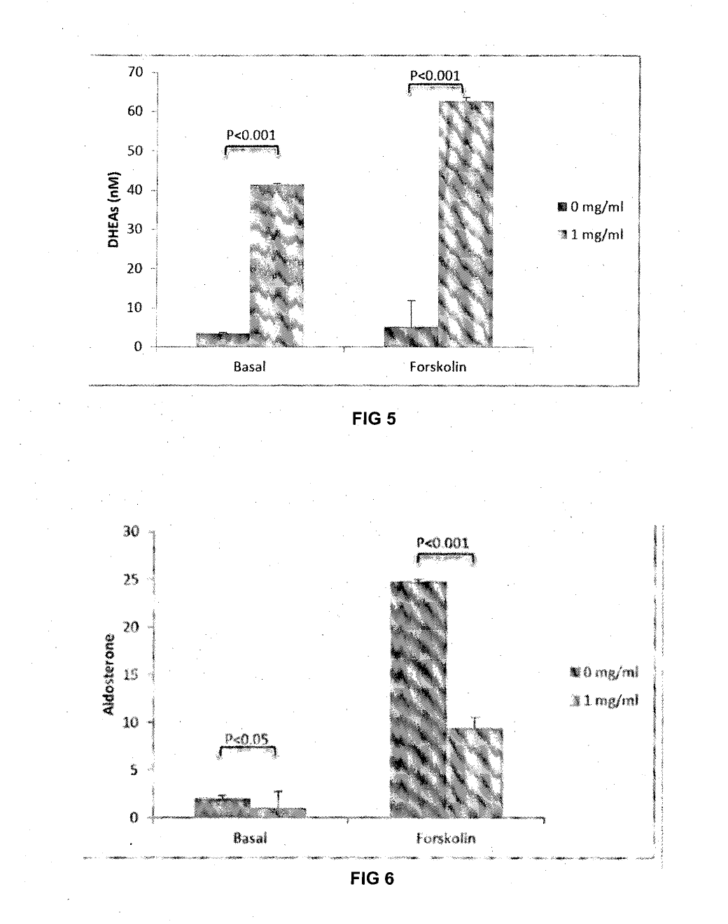

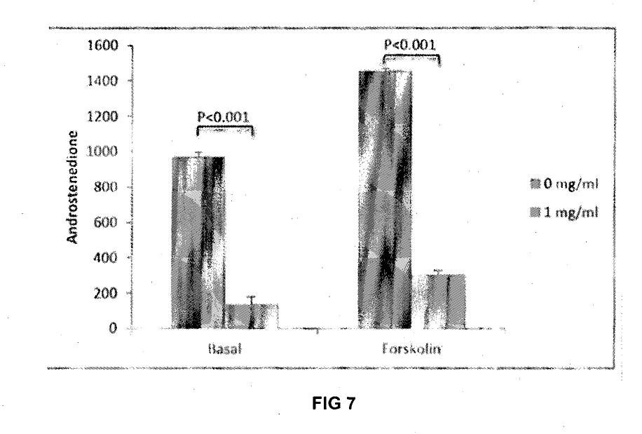

### BACKGROUND OF THE INVENTION

THIS invention relates to a composition comprising as an active ingredient an extract of a plant from the Mesembryanthemaceae family, in particular Mesembryanthemum tortuosum (Sceletium tortuosum), having a particular alkaloid profile such that the major component thereof is a combination of mesembrine, Δ7 mesembrenone, and mesembrenone, and its use as a bioamine releasing agent, in particular as a monoamine releasing agent (MRA) of the selective type.

Mesembryanthemum tortuosum, or Sceletium tortuosum as it is more commonly referred to in modern times, has been used for many centuries by indigenous peoples of Southern Africa, most notably the southern parts of the Western Cape and Namaqualand, and its use has been recorded in the literature for over 300 years. When prepared for chewing, typically by crushing of selected parts of the plant material, fermentation and drying, the resultant product, known locally as “kougoed” (stuff to chew), “Channa” or “Kanna”, is said to have mood enhancing and stimulant properties, and even pain and hunger relieving properties.

U.S. Pat. No. 6,288,104 discloses the use of mesembrine and related compounds as serotonin-uptake inhibitors, which can be used in the treatment of mild to moderate depression, psychological and psychiatric disorders where anxiety is present, major depressive episodes, i.e. single episode and recurrent depression with associated anxiety, in alcohol and drug dependence, in the treatment of bulimia nervosa, and in the treatment of obsessive-compulsive disorders. However in extensive studies conducted in respect of the present invention, it has been shown that, contrary to popular belief, mesembrine and its related alkaloids do not show serotonin-uptake inhibition properties.

US patent publication 2012/0004275 discloses compositions including as active ingredient an extract of a plant of the family Mesembryanthemaceae with mesembrenol and mesembrenone as the two major alkaloids present, and to their use as PDE4 inhibitors. In the disclosure it is pointed out that it is generally believed that plants of the genus Sceletium, and extracts thereof, should preferably contain high concentrations of mesembrine to contribute substantially to the known biological activity thereof. However, it is also noted that mesembrine has been reported to be unstable under a variety of conditions that can occur while harvesting, drying, and extracting the raw material, as well as during storage and formulation of the extract. It notes further that mesembrine has been shown to be unstable under conditions of fermentation, exposure to light, exposure to heat, and in an aqueous medium (citing Patnala, S. and Kanfer, I. Investigations of the phytochemical content of Sceletium tortuosum following the preparation of “Kougoed” by fermentation of plant material. J. Ethnopharmacol. 2009 Jan. 12; 121 (1):86-91).

### SUMMARY OF THE INVENTION

According to a first aspect of the invention, there is provided a composition comprising as active ingredient an extract of a plant or plants from the Mesembryanthemaceae family, such as Mesembryanthemum tortuosum, having an alkaloid profile comprising at least 60%, or at least 65%, or at least 70% (w/w) stabilized mesembrine.

According to a second aspect of the invention, there is provided a composition comprising as active ingredient an extract of a plant or plants from the Mesembryanthemaceae family, such as Mesembryanthemum tortuosum, having an alkaloid profile comprising at least 70% (w/w) mesembrine, preferably stabilized mesembrine, and the majority of the balance being Δ7 mesembrenone and mesembrenone.

In one form of this aspect of the invention the composition comprises at least about 70% (w/w) stabilized mesembrine, about 20% (w/w) mesembrenone and about 10% (w/w) Δ7 mesembrenone.

According to a third aspect of the invention, there is provided the use of a composition comprising as active ingredient an extract of a plant or plants from the Mesembryanthemaceae family, such as Mesembryanthemum tortuosum, the active ingredient comprising as a bioamine releasing agent, in particular as a monoamine releasing agent, at least 70% (w/w) stabilized mesembrine, for treating a patient having a disease or condition responsive to treatment with a bioamine releasing agent, in particular a monoamine releasing agent.

According to a fourth aspect of the invention, there is provided the use of a composition comprising as active ingredient an extract of a plant or plants from the Mesembryanthemaceae family, such as Mesembryanthemum tortuosum, in the manufacture of a medicament comprising as a bioamine releasing agent, in particular as a monoamine releasing agent, at least 70% (w/w) stabilized mesembrine for treating a patient having a disease or condition responsive to treatment with a bioamine releasing agent, in particular a monoamine releasing agent.

According to a fifth aspect of the invention, there is provided a method of treating a patient having a disease or condition responsive to treatment with a bioamine releasing agent, in particular a monoamine releasing agent, the method comprising administering to a patient in need of such treatment a composition comprising as active ingredient an extract of a plant or plants from the Mesembryanthenumaceae family, such as Mesembryanthemum tortuosum, the active ingredient comprising as a bioamine releasing agent, in particular as a monoamine releasing agent, at least 70% (w/w) stabilized mesembrine.

In some embodiments of the invention, the monoamine releasing agent is a serotonin-releasing agent.

In some embodiments of the invention, the compositions are provided as pharmaceutical compositions, whilst in other embodiments of the invention they are provided as dietary or other supplements, for example.

In the case of pharmaceutical compositions, in some embodiments of the invention they are provided in unit dosage form, each unit dose containing from about 100 μg to about 2.5 mg, or from about 300 μg to about 2 mg, or more preferably from about 500 μg to about 1.5 mg active ingredient.

In the case of dietary supplements, in some embodiments of the invention they comprise, per serving, from about 50 μg to about 800 μg, or from about 200 μg to about 600 μg, or from about 150 μg to about 250 μg active ingredient.

In some embodiments of the invention, the disease or condition responsive to treatment with a biomaine releasing agent, in particular a monoamine releasing agent, is selected from the group comprising cancer, mild to moderate depression, stress or anxiety, inflammation, obesity, hypertension and obsessive-compulsive disorder.

In some embodiments of the invention, the active ingredient comprises an extract from the plant Mesembryanthemum tortuosum, in particular of the DV-17 variety.

### DESCRIPTION OF PREFERRED EMBODIMENTS

A composition comprising as active ingredient an extract of a plant or plants from the Mesembryanthemaceae family, such as Mesembryanthemum tortuosum, in particular of the DV-17 variety, has an alkaloid profile comprising at least 70% (why) stabilized mesembrine.

Whilst extracts from the plant(s) of the Mesembryanthemaceae family have previously been identified as serotonin-uptake inhibitors, it has now surprisingly been found that extracts from Mesembryanthemum tortuosum having an alkaloid profile comprising at least about 70% (w/w) stabilized mesembrine, about 20% (w/w) mesembrenone and about 10% (w/w) Δ7 mesembrenone act as bioamine releasing agents, in particular as a monoamine releasing agent useful in the treatment of diseases and conditions responsive to treatment with a monoamine releasing agent, for instance diseases and conditions responsive to treatment with a serotonin-releasing agent.

It has also surprisingly been found that, whilst traditionally mesembrine has been found to be unstable under various conditions and hence commercially not viable as an active ingredient for pharmaceutical use, the mesembrine extracted and prepared in accordance with the method of the present invention has been found to be sufficiently stable for pharmaceutical use.

By “stabilized mesembrine” is meant that a composition comprising the mesembrine extracted according to a method of the present invention is stable for a period of at least 18 months if kept cool, dry, and away from any light source.

“The Variety DV-17” is a unique variety of Mesembryanthemum (Sceletium) tortuosum selectively propagated for its high alkaloid content, recognizable profile and vigorous growth. Chemical analysis shows a distinctive fingerprint to the levels of Δ7 mesembrenone, mesembrenone, mesembrine, and epimesembranol as well as other active and related compounds pre-fermentation.

Examples below are of 6 commercially available mesembryanthemum plants showing distinct alkaloid profiles unlike that of the DV-17 variety.

| Plant | Mesembrenone & | | |
|---|---|---|---|
| sample Ref | Mesembrenol % | Mesembranol % | Mesembrine % |
| WH-1 | 50.6 | 46.3 | 3.1 |
| WH-2 | 25.3 | 72.5 | 2.2 |
| WH-3 | 95.7 | 4.1 | 0.2 |
| WH-4 | 78.3 | 2.2 | 19.5 |
| DV-8 | 40.2 | 3.8 | 56.0 |
| DV-12 | 45.8 | Trace | 54.2 |

Yield figures for mesembrine are typically between 12 mg and 15 mg per gram of dried DV-17 whole herba.

The first aspect of the invention is a composition that comprises an extract of a plant or plants from the Mesembryanthemaceae family, such as Mesembryanthemum tortuosum, as active ingredient. It has an alkaloid profile comprising at least 60% (w/w) or at least 65% (w/w) or at least 70% (w/w) stabilized mesembrine.

The second aspect of the invention is a composition having an alkaloid profile comprising at least 70% (w/w) mesembrine, preferably stabilized mesembrine, and the majority of the balance being mesembrenone and Δ7 mesembrenone.

Such a composition typically comprises at least about 70% (w/w) stabilized mesembrine, about 20% (w/w) mesembrenone and about 10% (w/w) Δ7 mesembrenone.

The third aspect of the invention is the use of a composition of the invention as defined above for treating a patient having a disease or condition responsive to treatment with a bioamine releasing agent, in particular a monoamine releasing agent.

The fourth aspect of the invention is the use of a composition of the invention as defined above in the manufacture of a medicament for treating a patient having a disease or condition responsive to treatment with a bioamine releasing agent, in particular a monoamine releasing agent.

The fifth aspect of the invention is a method of treating a patient having a disease or condition responsive to treatment with a bioamine releasing agent, in particular a monoamine releasing agent. The method comprises administering to a patient in need of such treatment a composition as defined hereinbefore.

In some embodiments of the invention the compositions are pharmaceutical compositions. In other embodiments of the invention they are provided as dietary supplements or the like.

The pharmaceutical compositions of the invention may be provided in unit dosage form, each unit dose containing from about 100 μg to about 2.5 mg, or from about 300 μg to about 2 mg, or from about 500 μg to about 1.5 mg active ingredient.

The dietary supplements of the invention may contain, per serving, from about 50 μg to about 800 μg, or from about 200 μg to about 600 μg, or from about 150 μg to about 250 μg active ingredient.

In some embodiments of the invention, the active ingredient comprises an extract from the plant Mesembryanthemum tortuosum variety DV-17.

In some embodiments of the invention the mesembrine and related compounds are isolated and incorporated into a composition of the invention. Mesembrine is preferably used as its (−)-isomer i.e (−)-mesembrine.

The compounds of the invention have a unique mode of action as a monoamine releasing agent and in combination with other Mesembryanthemum compound isolates at specified doses may act as a short acting stimulant, mild europhorigenic, anti-depressant, anti-psychotic, minor tranquilizer and anxiolytic.

Thus the compositions of the invention may be useful in the treatment of diseases or conditions selected from the group consisting of mild to moderate depression, stress and anxiety, cancer, inflammation, obesity, hypertension and obsessive-compulsive disorders.

The compositions of the invention may be formulated in any suitable form for pharmaceutical administration, such as for example aqueous-ethanolic tinctures, tablets, capsules, nasal sprays, and as propylene glycol solutions. The formulations may be designed for use orally, sublingually, intra-nasally, transdermally, and via the pulmonary route by means of an electronic vaporiser.

The preferred compounds for the pharmaceutical compositions and methods of their use are (−)-mesembrine, Δ7 mesembrenone, and mesembrenone.

The pharmaceutical composition of the invention may comprise a hydro-methanolic extract of the plant containing desired amounts of mesembrine, Δ7 mesembrenone, or mesembrenone. Accordingly, the pharmaceutical compositions, whilst derived from a natural plant material, must contain a known and specified content of the active components.

An important aspect of the present invention is that the mesembrine is stable, which is due to its method of extraction and preparation.

### Adsorbent Preparation

10 kg of magnesium silicate, mesh finer than 200; 60 A; standard (650° C.) is weighed in a suitable container and is washed with 20 litres of 8% ammonia solution followed by 30 litres of DiH₂O. The adsorbent is then heated at 104° C. until a uniform moisture content of between 14-16%, preferably 15%, is reached to form an adsorbent/stationary phase.

### Plant extract

Plant (Mesembryanthemum tortuosum DV-17 variety) is harvested and dried (NMT8% moisture) then milled to 250 micron. 2 kg of said plant material is added to 10 litres of methanol (82% hydro-methanolic) at 35° C. with the PH adjusted to 4.4 with citric acid then allowed to stir for 24 hrs. The crude solvent extract is removed by pressing before an additional 2 litres of methanol 88% is again added to the plant material—this time without the addition of acid—and stirred for 9 hrs before repressing.

The combined crude extracts (9.8 litres) are filtered thrice through polypropylene membranes—1st 100 μm, 2nd 40 μm, and 3rd 8 μm.

7.8 litres of the main solvent (methanol) is recovered under reduced pressure—318 mb at 40° C. 1.5 kg of Kaolin powder (light) and 2.5 kg of the adsorbent/stationary phase are combined and added over 1 hour, in five equal parts, to the aqueous extract (1.52 litres) with constant stirring. It is very important that the temperature is kept below 17° C. during this addition stage as the evolution of excessive heat during the reaction will result in decreased yields due to permanent adhesion to the stationary phase as well as the formation of undesirable artifacts. The resulting paste is dried under vacuum and then reduced to a free flowing powder of 90 μm.

### Isolation of Actives

360 g of the above extract/powder is packed into a Luknova flash chromatography cartridge. The column is loaded onto a MPLC (Grace, Reveler's) instrument, set and run as follows:

Cartridge: Luknova 360 g reusable

Equilibration: 1 CV

Flow rate: 80 mL/min

UV threshold: 0.03 AU

UV1 wavelength: 220 nm

UV2 wavelength: 254 nm

ELSD threshold: 5 mv

ELSD carrier: Iso-proponal

Set peaks: Collect all

Injection type: n/a

Solvent A: Cyclohexane

Solvent B: DCM

Solvent C: Acetone

Solvent D: Ethanol/25% Ammonium hydroxide solution (95:5)

Mobile phase: A:B/B:C/C:D

Run time: 72 min

Combine fractions of interest 7 to 11 (as confirmed by TLC)—Evaporate solvent under reduced pressure to obtain the purified alkaloid fraction of 70% mesembrine/20% mesembrenone/≦10% Δ7 mesembrenone. Fractions 12+13 contain mainly the alkaloid epimesembranol.

### Spray Drying

Combine the following in a suitable container:

14 g alkaloid containing composition

807 ml DiH₂O at 35° C.

Adjust pH with citric acid to 5.4

Add 5 g Gum Acacia and 250 g Mannitol to the alkaloid solution under high sheer conditions until homogenized and then spray dry under the following conditions:

Model: LabTex—M1 Spray drier

Inlet temperature: 158° C.

Outlet temperature: 102° C.

Pump speed: 12 mL/min

Feed solution: Total solids at 25% w/w

Nozzle pressure: 0.6 bar

Aspirator speed: 9 (75 cu.m/hr)

Yield: 236 g

Product Characteristics: Free flowing powder

Colour: Tan

Practical Size: 177 μm (fines)

Moisture Content: <3%

Total Alkaloids: NLT5% w/w

Alkaloid Profile: Mesembrine 70%, Mesembrenone 20%, Δ7

mesembrenone ≦10%

Product is stable for 18 months if kept cool, dry, and away from any light source.

### Thin-Layer Chromatography

For qualitative screening purposes the following system is suitable: AnalTech, Inc RPS-F Silica Gel W/UV254 (250 μm layer thickness) and developed in water/methanol/ammonia solution N7, in methanol (18:6:0.5). The plates are dried at 60° C. for 10 minutes, studied under UV254 and UV365 and then sprayed with Dragendorff's spray reagent.

(Rf of mesembrine=0.38)

### High Performance Liquid Chromatography (HPLC)

The alkaloids of the invention may directly be extracted in ethanol, methanol or any other suitable solvent. For HPLC the sample has to be filtered (e.g. 0.45 μm filter) in order to protect the columns from impurities.

Separation of the stabilized extract using a mobile phase comprising of water:acetonitrile:ammonium hydroxide solution mixed in a ratio of 72:28:0.01 (v:v:v).

Column—Hypersil® 150×4.6 mm i.d, C18 column (Phenomenex®, Torrence, Calif., USA).

The effects of the stabilized mesembrine compositions of the invention were evaluated in a number of non-exhaustive studies.

### A. Effect of the Stabilized Mesembrine Composition on Viability of Adrenal Cells in Culture

Viability tests were conducted on all cell types prior to use in experiments. The first study was conducted using adrenal cells (H295R cell line).

Briefly, the procedure entailed 48 hour incubation of adrenal cells in the presence of both low (0.0001 mg/ml) and high (1 mg/ml) concentrations of the stabilized mesembrine composition after which the viability of cells were assessed using the Trypan blue method. (Live cells will not take up the Trypan blue dye, but it readily diffuses into dead or compromised cells.) In addition, viability was also assessed in a simulated stress condition, using forskolin (explanation below in part B).

Results are illustrated below. Results are expressed as the percentage of cells still viable after 48 hours. Bars are means of repeated experiments (a minimum of 4 repeats) and error bars indicate standard deviations.

As is evident from the above graph, the stabilized mesembrine composition (“Tri”) had no adverse effect on cell viability. In fact, viability of cells was significantly better after incubation in the presence of the stabilized mesembrine composition (stats: one-way ANOVA with Bonferroni post hoc tests), as indicated on the graph.

### B) Effect of the Stabilized Mesembrine Composition on in vitro Steroidogenesis

The effect of the stabilized mesembrine composition on steroid synthesis using in vitro techniques was investigated. The H295R cell line is an adrenal cell type, uniquely able to produce 22 steroid hormones and steroid metabolites, with which the steroid synthesis pathway may be comprehensively mapped. Various concentrations of the stabilized mesembrine composition were added to these cells to investigate its effect under basal (normal) conditions. In addition, cultures were stimulated with forskolin, which forces glucocorticoid biosynthesis—thereby simulating a condition of stress. This allowed assessment of effects of the stabilized mesembrine composition in a stressed condition. Main results are illustrated below, following the format used for reporting the viability studies.

One-way ANOVA and Newman-Keuls post hoc testing was performed to compare concentrations of steroid hormones and metabolites synthesized in the presence or absence of the stabilized mesembrine composition. In addition, data obtained after additional forskolin stimulation—representing effects in stressed conditions—are presented by the second set of bars in each graph. For clarity, only the results obtained when using the highest dose of the stabilized mesembrine composition were chosen for this study (1 mg/ml). For the same reason, only statistical results relevant to this report are indicated on the graphs.

Several important effects were observed. These will be discussed below the graphical illustration of each result.

### Cortisol

Addition of forskolin greatly increased cortisol synthesis as anticipated, since forskolin forces glucocorticoid synthesis, which is expected in a condition of stress. In both unstressed (basal) and stressed (forskolin) conditions addition of 1 mg/ml significantly decreased adrenal cortisol synthesis. This was most likely the result of suppression of some adrenal enzymes vital to the steroid synthesis pathway, since the level of relevant hormone metabolites were also decreased (data not shown).

### DHEAs

DHEAs (dehydroepiandrosterone-sulphate) is a known antagonist to cortisol. The fact that the stabilized mesembrine composition facilitated an increase in the synthesis of this hormone is very encouraging. This data very importantly indicates that the anti-stress effect observed in the cortisol data was not the result of a blanket inhibition of cell metabolism by the stabilized mesembrine composition, but a true modulation of the steroid synthetic pathway—i.e. it indicates specificity of action of the stabilized mesembrine composition. The DHEAs data specifically has many implications for application as therapeutic intervention, including, as cortisol antagonist, the increased DHEAs synthesis suggest an even more efficient anti-stress action, and a higher DHEAs level has been linked to slower cognitive deterioration and improved memory.

### Aldosterone

In terms of the aldosterone data, the stabilized mesembrine composition significantly decreased aldosterone levels. Since increased aldosterone is one of the major causes of hypertension, decreased levels suggest that the stabilized mesembrine composition might lower blood pressure.

### Androstenedine

The final result is that of Androstenedione synthesis. An increased level of this hormone is associated with pathology of the prostate, so again this result has potential for huge impact in the treatment and protection of prostate cancer.

Examples of pharmaceutical compositions of the invention are provided for illustrative purposes and are not intended to be limiting on the scope of the invention.

### EXAMPLE 1

A liquid composition comprises of 65% vegetable glycerine/20% ethanol/10% water solvent containing about 500 μg/ml of the composition of the invention.

A typical oral dose of the liquid composition is from 1 ml to 5 ml inclusive daily.

### EXAMPLE 2

A sublingual tablet contains 500 μg of a composition of the invention, and conventional pharmaceutical excipients.

A typical dose of the sublingual tablet composition is from 1 to 5 inclusive daily.

### EXAMPLE 3

An oral tablet contains 2.5 mg of a composition of the invention, and conventional pharmaceutical excipients.

A typical dose of the oral tablet composition is from 1 to 2 inclusive daily.

### EXAMPLE 4

A transdermal hydro-gel composition comprises of 80% water/16% ethanol/1% guar gum/1% gum Arabic/1% DMSO containing about 150 mg/ml of a composition of the invention.

A typical dose of the hydro-gel composition is from 0.1 ml to 0.5 ml inclusive daily.

### EXAMPLE 5

A pharmaceutical strength oral tablet contains 15 mg of a composition of the invention, and conventional pharmaceutical excipients.

A typical dose of the oral tablet composition is from 1 to 4 inclusive daily under the supervision of a medical practitioner.

### EXAMPLE 6

A pharmaceutical strength liquid composition comprises 65% propylene glycol/25% vegetable glycerine/10% water/5% ethanol/citric acid solvent calculated to contain about 160 mg/ml of the composition of the invention after the filtration of all insoluble solids.

A typical dose of the liquid to vapour composition—by means of a suitable device capable of producing said vapour as micro-droplets, for example—is from 25 μl to 150 μl inclusive daily under the supervision of a medical practitioner.

### EXAMPLE 7

A pharmaceutical strength liquid composition comprises 55% sterile deionised water/36% vegetable glycerine/7% sodium chloride/1.5% sorbitol powder/0.1% potassium sorbate/citric acid solvent containing about 40 mg/ml of the composition of the invention.

A typical dose of the liquid to spray composition—by means of a suitable device capable of producing said spray as micro-droplets e.g. nasal jet-spray pump—is from 500 μl to 6 ml inclusive daily under the supervision of a medical practitioner.

1. A composition comprising as active ingredient an extract of a plant or plants from the Mesembryanthemaceae family having an alkaloid profile comprising at least 60% (w/w) stabilized mesembrine.

2. A composition according to claim 1, comprising at least 65% (w/w) stabilized mesembrine.

3. A composition according to claim 1, comprising at least 70% (w/w) stabilized mesembrine.

4. A composition comprising as active ingredient an extract of a plant or plants from the Mesembryanthemaceae family having an alkaloid profile comprising at least 70% (w/w) mesembrine, and the majority of the balance being Δ7 mesembrenone and mesembrenone.

5. A composition according to claim 4, comprising at least about 70% (w/w) stabilized mesembrine.

6. A composition according to claim 5, comprising at least about 70% (w/w) stabilized mesembrine, about 20% (w/w) mesembrenone and about 10% (w/w) Δ7 mesembrenone.

7. Use of a composition comprising as active ingredient an extract of a plant or plants from the Mesembryanthemaceae family, the active ingredient comprising as a bioamine releasing agent, in particular as a monoamine releasing agent, at least 70% (w/w) stabilized mesembrine, for treating a patient having a disease or condition responsive to treatment with a bioamine releasing agent, in particular a monoamine releasing agent.

8. Use of a composition comprising as active ingredient an extract of a plant or plants from the Mesembryanthemaceae family, in the manufacture of a medicament comprising as a bioamine releasing agent, in particular as a monoamine releasing agent, at least 70% (w/w) stabilized mesembrine for treating a patient having a disease or condition responsive to treatment with a bioamine releasing agent, in particular a monoamine releasing agent.

9. A method of treating a patient having a disease or condition responsive to treatment with a bioamine releasing agent, in particular a monoamine releasing agent, the method comprising administering to a patient in need of such treatment a composition comprising as active ingredient an extract of a plant or plants from the Mesembryanthenumaceae family, the active ingredient comprising as a bioamine releasing agent, in particular as a monoamine releasing agent, at least 70% (w/w) stabilized mesembrine.

10. A method according to claim 9, wherein the monoamine releasing agent is a serotonin-releasing agent.

11. A method according to claim 9, wherein the disease or condition responsive to treatment with a monoamine releasing agent is selected from the group comprising cancer, mild to moderate depression, stress or anxiety, inflammation, obesity, hypertension and obsessive-compulsive disorder.

12. A composition according to claim 1, which is provided as a pharmaceutical composition.

13. A composition according to claim 12, which is provided in unit dosage form, each unit dose containing from about 100 μg to about 2.5 mg, or from about 300 μg to about 2 mg, or from about 500 μg to about 1.5 mg active ingredient.

14. A composition according to claim 1, which is provided as a dietary or other supplement.

15. A composition according to claim 14, comprising, per serving, from about 50 μg to about 800 μg, or from about 200 μg to about 600 μg, or from about 150 μg to about 250 μg active ingredient.

16. A composition according to claim 1, wherein the active ingredient comprises an extract from the plant Mesembryanthemum tortuosum.

17. A composition according to claim 1, wherein the active ingredient comprises an extract from the plant Mesembryanthemum tortuosum of the DV-17 variety.
